(19) United States
(12) Patent Application Publication
(10) Pub. No.: US 2021/0100932 A1
(43) Pub. Date: Apr. 8, 2021

(54) ALIGNED ELECTROSPUN MATRICES OF DECELLULARIZED MUSCLE FOR TISSUE REGENERATION

(71) Applicant: Saint Louis University, St. Louis, MO (US)

(72) Inventors: Koyal Garg, St. Louis, MO (US); Scott A. Sell, St. Louis, MO (US); Krishna Patel, Schaumburg, IL (US); Hady Elmashhady, St. Louis, MO (US); Emily Kalaf, St. Louis, MO (US)

(21) Appl. No.: 16/499,384

(22) PCT Filed: Mar. 30, 2018
(86) PCT No.: PCT/US2018/025402

§ 371 Date: Sep. 30, 2019

Related U.S. Application Data
(60) Provisional application No. 62/478,728, filed on Mar. 30, 2017.

(51) Int. Cl. A61L 27/36; A61L 27/58; A61L 27/38; A61L 27/18; D01F 8/18; D01F 8/14

(52) U.S. Cl. CPC A61L 27/3691; A61L 27/3633; A61L 27/58; A61L 27/3826; A61L 27/18; D10B 2509/00; D01F 8/18; D01F 8/14; A61L 2430/30; A61L 2400/12; A61L 27/3873

(57) ABSTRACT

Disclosed are electrospun scaffolds and methods of making electrospun fibers and electrospun fiber scaffolds. More particularly, the present disclosure relates to electrospun fibrous scaffolds of decellularized muscle tissue and methods of making fibers and fiber scaffolds by electrospinning.

### CROSS REFERENCE TO RELATED APPLICATIONS

This application claims priority to U.S. Provisional Application No. 62/478,728, filed on Mar. 30, 2017, the disclosure of which is hereby incorporated by reference in its entirety.

### BACKGROUND OF THE DISCLOSURE

The present disclosure relates generally to electrospun scaffolds and methods of making fibers and fibers scaffolds by electrospinning More particularly, the present disclosure relates to electrospun fibrous scaffolds of decellularized muscle tissue and methods of making fibers and fiber scaffolds by electrospinning

A majority of extremity injuries sustained in vehicular accidents and military conflicts involve severe musculoskeletal trauma. Skeletal muscle possesses a remarkable ability to repair and regenerate following minor injuries. However, when considering frank loss of muscle tissue (>20%) termed as a volumetric muscle loss (VML) injury, the muscle tissue is unable to repair and regenerate itself. As a result, VML can contribute to military medical discharge, long-term disability, low quality of life and delayed or elected limb amputation.

Currently, there is no definitive therapy for VML. Thus far, implantation of decellularized extracellular matrix (D-ECM) scaffolds into VML injury sites have failed to appreciably regenerate muscle tissue and often result in fibrotic tissue deposition. Poor mechanical strength and absence of stem cell migration into D-ECM scaffolds are the primary reasons for impaired muscle regeneration.

Accordingly, there exists a need for materials and methods for muscle tissue regeneration.

### BRIEF DESCRIPTION OF THE DISCLOSURE

The present disclosure is generally directed to electrospun nanofibrous scaffolds and methods of making fibers and fiber scaffolds by electrospinning More particularly, the present disclosure relates to electrospun nanofibrous scaffolds of decellularized muscle tissue and methods of making fibers and fiber scaffolds by electrospinning

In one aspect, the present disclosure is directed to an electrospun decellularized-extracellular matrix (D-ECM) fiber.

In one aspect, the present disclosure is directed to an electrospun decellularized-extracellular muscle matrix (D-ECM matrix) scaffold.

In one aspect, the present disclosure is directed to a method for preparing an electrospun decellularized-extracellular muscle matrix (D-ECM matrix) scaffold. The method includes decellularizing a striated muscle tissue to prepare a D-ECM matrix; and electrospinning the D-ECM matrix to prepare a D-ECM matrix scaffold.

In accordance with the present disclosure, materials and methods have been discovered that surprisingly allow for the isolation and identification of weakly interacting molecules from a fluidic sample using immiscible phase filtration. The methods of the present disclosure have a broad and significant impact, as they allow interactions between molecules that were previously unidentifiable using traditional methods to be identified, and a “snapshot” of the molecular interactions at (or close to) equilibrium to be obtained. This is not possible with traditional methods that use aqueous wash steps, as equilibrium is perturbed with each wash step, which results in the loss of weakly interacting molecules.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 are scanning electron micrographs of anisotropic and isotropic electrospun scaffolds composed of pure PCL, hybrid PCL and D-ECM and pure D-ECM.

FIGS. 2A-2C are graphs depicting (FIG. 2A) fiber-diameter, (FIG. 2B) pore-radius and (FIG. 2C) porosity of the electrospun scaffolds.

FIGS. 3A-3C depict pixel intensity plots against the angle of acquisition for the aligned and random fibers in the electrospun scaffolds for 10% PCL (FIG. 3A), 5% PCL+5% D-ECM (FIG. 3B), and 10% D-ECM (FIG. 3C).

FIGS. 4A-4D are graphs depicting (FIG. 4A) peak load, (FIG. 4B) peak stress, (FIG. 4C) modulus and (FIG. 4D) strain at break of anisotropic and isotropic electrospun scaffolds.

FIG. 5 depicts satellite cells seeded on electrospun scaffolds and stained with desmin (green channel) and DAPI (blue channel) to visualize nuclei.

FIG. 6A depicts SDS-PAGE of D-ECM for collagen α1 and α2 chains and several lower molecular weight proteins.

FIG. 6B depicts Western blotting analysis of bands in FIG. 6A that stained positive for collagen type 1 and laminin γ1 chain in the D-ECM as well as the electrospun scaffolds.

FIGS. 7A and 7B depict quantification of VEGF in cell culture supernatants (FIG. 7A) and IL-6 (FIG. 7B).

FIGS. 8A-8D depict quantification of cellular protein lysates for myogenic proteins. FIG. 8A depicts Western blot analysis of cellular protein lysates for myogenic proteins. FIG. 8B depicts quantification of MyoD expression in the aligned PCL:D-ECM compared to the aligned PCL. FIG. 8C depicts quantification of myogenin in the aligned PCL:D-ECM compared to the aligned PCL. FIG. 8D depicts quantification of α-actinin in the aligned PCL:D-ECM compared to the aligned PCL. #difference between random and aligned; difference between D1-D4.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used herein, “electrospinning” or “electrospun,” refers to any method where materials are streamed, sprayed, sputtered, dripped, or otherwise transported in the presence of an electric field. The electrospun material can be deposited from the direction of a charged container towards a grounded target, or from a grounded container in the direction of a charged target. In particular, the term “electrospinning” refers to the formation of fibers from a charged solution that includes a decellularized-extracellular matrix (D-ECM matrix). The charged solution can further include at least one synthetic polymer material. The electrically charged solution is then streamed through an opening or orifice towards a grounded target. As known to those skilled in the art, electrospinning generally involves the creation of an electrical field at the surface of a liquid. The resulting electrical forces create a jet of liquid which carries electrical charge. As the jet of liquid elongates and travels, it will harden and dry to produce fibers.

As used herein, “decellularized” or “decellularizing” as used herein refers to the removal of cellular components from a tissue (e.g., muscle) leaving behind an intact acellular infra-structure. The acellular infra-structure represents the complex three-dimensional network of connective tissue that is primarily composed of collagen. The decellularized matrix provides a biocompatible substrate onto which different cell populations can be infused. The decellularized matrix can be rigid and semi-rigid.

In one aspect, the present disclosure is directed to an electrospun decellularized-extracellular muscle matrix (D-ECM matrix) fiber.

The diameter of the electrospun D-ECM matrix fiber can range from about 50 nanometers to about 2 μm, including from about 1 μm to about 2 μm.

The electrospun D-ECM matrix fiber can further include a polymer. Suitable polymers can be chosen from polycaprolactone, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol) (PVA), poly(acrylic acid), poly(vinyl acetate), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), poly(vinyl acetate), polyvinylhydroxide, poly(ethylene oxide) (PEO) and polyorthoesters, and combinations thereof.

The D-ECM matrix can be combined with a polymer material to form electrospun fibers including D-ECM matrix and polymer. The D-ECM matrix and/or polymer material can be dissolved or suspended in a solution or suspension in water, urea, methanol, chloroform, monochloroacetic acid, isopropanol, 2,2,2-trifluoroethanol, 1,1,1,3,3,3-hexafluoro-2-propanol (also known as hexafluoroisopropanol or HFP), acetamide, N-methylformamide, N,N-dimethylformamide (DMF), dimethylsulfoxide (DMSO), dimethylacetamide, N-methyl pyrrolidone (NMP), acetic acid, trifluoroacetic acid, ethyl acetate, acetonitrile, trifluoroacetic anhydride, 1,1,1-trifluoroacetone, maleic acid, hexafluoroacetone and combinations thereof.

The electrospun D-ECM matrix fiber can include from about 0.1% D-ECM matrix by weight to 100% D-ECM matrix by weight, including about 0.5% by weight, including about 1% by weight, including about 1.5% by weight, including about 5% by weight, including about 10% by weight, including about 20% by weight, including about 25% by weight, including about 30% by weight, including about 40% by weight, including about 50% by weight, including about 60% by weight, including about 70% by weight, including about 75% by weight, including about 80% by weight, including about 90% by weight, and including about 95% by weight.

The electrospun polymer fiber can include from about 0.1% polymer by weight to 100% polymer by weight, including about 0.5% by weight, including about 1% by weight, including about 1.5% by weight, including about 5% by weight, including about 10% by weight, including about 20% by weight, including about 25% by weight, including about 30% by weight, including about 40% by weight, including about 50% by weight, including about 60% by weight, including about 70% by weight, including about 75% by weight, including about 80% by weight, including about 90% by weight, and including about 95% by weight.

To form electrospun fibers having D-ECM matrix and polymer, D-ECM matrix can be mixed with a polymer solution at varying ratios of D-ECM matrix and polymer material.

Suitable polymers can be biodegradable, non-biodegradable and combinations thereof.

The electrospun decellularized-extracellular matrix (D-ECM matrix) fiber is made by decellularizing muscle tissue. Suitable detergents for decellularizing muscle tissue include TRITON™ X-100, TRITON™ N-101, TRITON™ X-114, TRITON™ X-405, TRITON™ X-705, TRITON™ DF-16, monolaurate (TWEEN® 20), monopalmitate (TWEEN® 40), monooleate (TWEEN® 80), and polyoxethylene-23-lauryl ether (BRIJ® 35), polyoxyethylene ether W-I (POLYOX™), sodium cholate, deoxycholates, CHAPS (3-[(3-Cholamidopropyl)dimethylammonio]-1-propanesulfonate), saponin, n-Decyl β-D-glucopuranoside, n-heptyl β-D glucopyranoside, n-Octyl-α-D-glucopyranoside and NONIDET™ P-40.

In one aspect, the present disclosure is directed to an electrospun decellularized-extracellular muscle matrix (D-ECM matrix) scaffold comprising an electrospun D-ECM matrix fiber.

The D-ECM matrix scaffold can further include a polymer fiber. Suitable polymers include polycaprolactone, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol) (PVA), poly(acrylic acid), poly(vinyl acetate), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), poly(vinyl acetate), polyvinylhydroxide, poly(ethylene oxide) (PEO) and polyorthoesters, and combinations thereof.

A polymer solution can be prepared and mixed with solubilized D-ECM matrix.

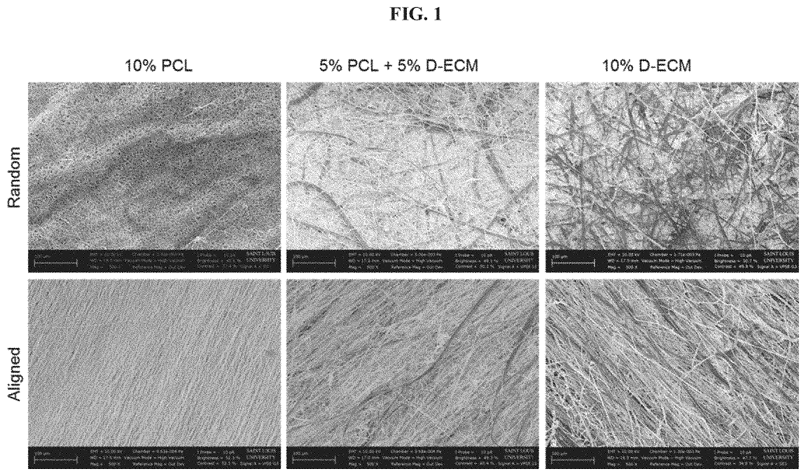

The D-ECM matrix scaffold can have randomly oriented D-ECM matrix fibers, aligned D-ECM matrix fibers, and combinations thereof as shown in FIG. 1.

The diameter of the electrospun D-ECM fiber can range from about 50 nanometers to about 2 μm, including from about 1 μm to about 2 μm.

The scaffolds can further include a cell. Particularly suitable cells include muscle cells.

The D-ECM matrix scaffolds can be used as tissue engineering scaffolds for wound healing, skeletal, cardiac or smooth muscle repair following ischemia or traumatic injury, and drug delivery.

In one aspect, the present disclosure is directed to a method for preparing an electrospun decellularized-extracellular muscle matrix (D-ECM matrix) scaffold. The method includes decellularizing a striated muscle tissue to prepare a D-ECM matrix; and electrospinning the D-ECM matrix to prepare a D-ECM matrix scaffold.

The muscle tissue is treated with detergents to remove the inhabiting cells. The resulting matrix is solubilized and electrospun to create scaffolds. Fibers of the scaffold can be aligned, randomly oriented, and combinations thereof. Aligned fibers are particularly desirable as they mimic the native architecture of striated muscle (e.g., skeletal muscle and cardiac muscle). The scaffold created using this technique has biologically relevant ratios of proteins and mimics both the native tissue composition and architecture.

Any striated muscle tissue is suitable for use in the method, including skeletal muscle tissue and cardiac muscle tissue.

Sources of skeletal muscle tissue include animal skeletal muscle, including human skeletal muscle.

The electrospun decellularized-extracellular matrix (D-ECM matrix) fiber is made by decellularizing muscle tissue to prepare a D-ECM matrix. Suitable detergents for decellularizing muscle tissue include TRITON™ X-100, TRITON™ N-101, TRITON™ X-114, TRITON™ X-405, TRITON™ X-705, TRITON™ DF-16, monolaurate (TWEEN® 20), monopalmitate (TWEEN® 40), monooleate (TWEEN® 80), and polyoxethylene-23-lauryl ether (BRIJ® 35), polyoxyethylene ether W-I (POLYOX™), sodium cholate, deoxycholates, CHAPS (3-[(3-Cholamidopropyl)dimethylammonio]-1-propanesulfonate), saponin, n-Decyl β-D-glucopuranoside, n-heptyl β-D glucopyranoside, n-Octyl-α-D-glucopyranoside and NONIDET™ P-40.

The decellularization solution can also include an alkaline solution. Suitable alkaline solutions include ammonium hydroxide ammonium sulphate, and ammonium acetate. Other alkaline solution consisting of ammonium salts or their derivatives can also be used.

The D-ECM matrix can be washed in deionized water to remove the detergent. Following the decellularization protocol, the D-ECM matrix can be frozen and lyophilized.

The D-ECM matrix can be digested and solubilized with a protease. A suitable protease includes pepsin.

To prepare the electrospun D-ECM matrix scaffold, the D-ECM matrix is dissolved or suspended in a solution or suspension in water, urea, methanol, chloroform, monochloroacetic acid, isopropanol, 2,2,2-trifluoroethanol, 1,1,1,3,3,3-hexafluoro-2-propanol (also known as hexafluoroisopropanol or HFP), acetamide, N-methylformamide, N,N-dimethylformamide (DMF), dimethylsulfoxide (DMSO), dimethylacetamide, N-methyl pyrrolidone (NMP), acetic acid, trifluoroacetic acid, ethyl acetate, acetonitrile, trifluoroacetic anhydride, 1,1,1-trifluoroacetone, maleic acid, hexafluoroacetone, and combinations thereof.

The electrospun D-ECM matrix scaffold can further include a polymer material by dissolving or suspending a polymer material in the same solution or suspension with the D-ECM matrix or a separate a solution or suspension as described herein.

The solution including the D-ECM matrix is electrically charged then streamed towards a grounded target and the electrospun D-ECM matrix scaffold is collected. The electrospun fibers can be collected as randomly oriented fibers and aligned fibers.

The scaffold has can be used in a number of tissue engineering applications as an acellular scaffold (i.e., skeletal muscle regeneration, wound healing, stem cell expansion, etc). The decellularized muscle matrix can also be combined with synthetic polymers such as poly(caprolactone) to improve the mechanical properties of the scaffold.

The average fiber diameter for the electrospun structure can be obtained by taking the average of measurements chosen randomly from across the image in ImageJ.

The electrospun D-ECM matrix scaffold including randomly oriented D-ECM matrix fibers can have a pore-radius ranging from about 20 μm to about 35 μm for 100% D-ECM scaffolds. The electrospun D-ECM matrix scaffold including randomly oriented D-ECM matrix fibers and PCL fibers can have a pore-radius ranging from about 2 μm to about 7 μm. The electrospun D-ECM matrix scaffold including aligned D-ECM matrix fibers can have a pore-radius ranging from about 5 μm to about 15 μm for 100% D-ECM scaffolds. The electrospun D-ECM matrix scaffold including aligned D-ECM matrix fibers and PCL fibers can have a pore-radius ranging from about 8 μm to about 12 μm.

The electrospun D-ECM matrix scaffold including randomly oriented D-ECM matrix fibers can have a porosity ranging from about 95% to about 99% for 100% D-ECM scaffolds. The electrospun D-ECM matrix scaffold including randomly oriented D-ECM matrix fibers and PCL fibers can have a porosity ranging from about 85% to about 95%. The electrospun D-ECM matrix scaffold including aligned D-ECM matrix fibers can have porosity ranging from about 85% to about 99%. The electrospun D-ECM matrix scaffold including aligned D-ECM matrix fibers and PCL fibers can have a porosity ranging from about 85% to about 99%.

The peak stress, peak load and strain at break can be adjusted by adjusting the concentration of D-ECM matrix in the scaffolds. For example, the peak stress, peak load and strain at break can be decreased by increasing the concentration of D-ECM matrix in the scaffolds.

The scaffolds can further include a cell. Particularly suitable cells include muscle cells.

The disclosure will be more fully understood upon consideration of the following non-limiting Examples.

### EXAMPLES

### Example 1

Decellularization of skeletal muscle and electrospinning of scaffolds

Skeletal muscle (including dorsal extensors, ventral and lateral flexors) was harvested from bovine tail. The tissue was subjected to a freeze thaw cycle following which the connective tissue and fat was removed. The muscle tissue was cut into 1 cm³ pieces and rinsed with deionized water for 24 hours at 4° C. under mechanical agitation. The tissue pieces were then stirred in decellularization solution (Triton X-100 and ammonium hydroxide (NH₄OH)) for 2-3 days at 4° C. The decellularized muscle was then stirred for 24 hours in deionized water to remove the detergent. Following the decellularization protocol, the muscle tissue was frozen and lyophilized to obtain the decellularized-extracellular matrix (“D-ECM matrix”). The D-ECM matrix was then digested and solubilized with pepsin, dialyzed against DI water, lyophilized and stored frozen at −80° C. until needed. Briefly, a solution of PCL with or without D-ECM in 1,1,1,3,3,3-hexafluoro-2-propanol (HFP) was loaded into a 10 mL syringe with a blunted needle. A 0.75-4 ml/hour flow rate was applied with a voltage of 20-25 kV, and a needle tip to collector plate distance of 10-15 cm. A rectangular, rotating mandrel (1000 rpm) was used as the grounded collector plate for collection of randomly oriented fibers. Alignment of fibers was achieved by rotating a larger, 3-D printed disc-shaped mandrel at a higher speed (4500 rpm). The larger diameter of the disc-shaped mandrel greatly increased surface velocity compared to the rectangular one. The disc-shaped mandrel was also modified with copper wires and a custom fabricated ring to concentrate electric charge to further aid in fiber alignment.

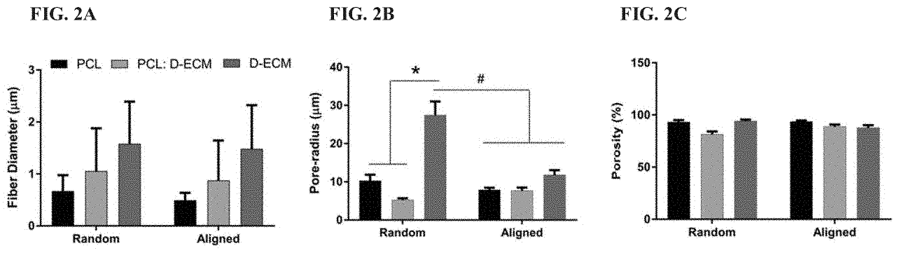

Scaffold characterization was performed using scanning electron microscopy (SEM) on small pieces cut from the electrospun mats. The SEM images show the feasibility of creating nanofibers from decellularized skeletal muscle alone or in combination with PCL (FIG. 1). Physical characterization of scaffolds included determining the average fiber diameter for the electrospun structure by taking the average of 60-100 measurements chosen randomly from across the image in ImageJ. The average fiber diameters of the scaffolds ranged from 1-2 μm (FIG. 2A). The 10 mm disks of the electrospun scaffolds were weighed and subsequently immersed in ethanol overnight with slight mechanical agitation. This was done to allow the ethanol to penetrate into the scaffold pores. The surface of the samples was then blotted dry on a filter paper and weighed once more to determine the mass of the ethanol present within the scaffold. Measurements were made on five sample disks of each scaffold type. The density of ethanol is 0.789 g/mL and the density of PCL is 1.34 g/mL. The porosity (ε) was calculated as:

$$ɛ = \frac{{v}_{ETH}}{{V}_{ETH} + {V}_{PCL}}$$

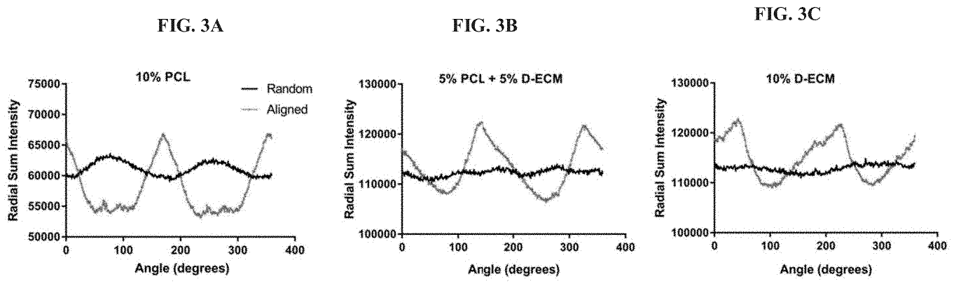

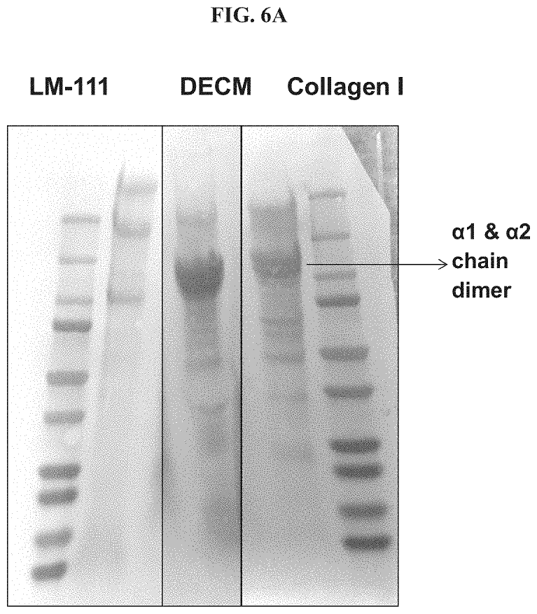

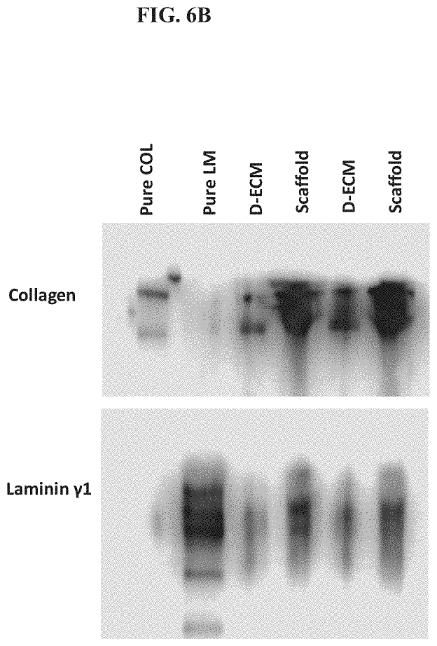

V_{ETH} is the volume of the intruded ethanol and was calculated as the ratio between the observed mass change after intrusion and ρ_{ETH}. V_{PCL} is the volume of PCL fibers and was calculated as the ratio between the dry scaffold mass before intrusion and the density of PCL (ρ_{PCL}) The pore-radius was calculated from the following equation:

$$r = \frac{ω}{ln ( \frac{1}{ɛ} )}$$

where ω is the fiber diameter and c is the scaffold porosity. The pore-radius of the 10% D-ECM scaffold with randomly oriented fibers was significantly higher than the 10% PCL and the composite 5% PCL and 5% D-ECM scaffold (FIG. 2B). The pore-radius of the all scaffolds with aligned fibers was also significantly lower than that of random 10% D-ECM scaffold. All scaffolds had high porosity but no significant differences between the scaffolds were noted (FIG. 2C). The Fast Fourier Transform (FFT) method was used to evaluate the fiber alignment of the electrospun scaffolds. The alignment of the fibers in the images was analyzed using the ImageJ. The pixel intensities were plotted between 0 to 360°, and the degree of alignment in the FFT data were reflected by the sharpness and heights of the peak shown on the plots for 10% PCL (FIG. 3A), 5% PCL+5% D-ECM (FIG. 3B), and 10% D-ECM (FIG. 3C). Scaffolds with aligned fibers showed sharper peaks that were fewer in occurrence and higher in intensity. SDS-PAGE of D-ECM revealed distinct bands for collagen α1 and α2 chains and several lower molecular weight proteins (FIG. 6A). Western blotting analysis revealed that these bands stained positive for collagen type 1 and laminin γ1 chain confirming the presence of these proteins in the D-ECM as well as the electrospun scaffolds (FIG. 6B).

### Example 2

In this Example, mechanical testing of scaffolds was determined.

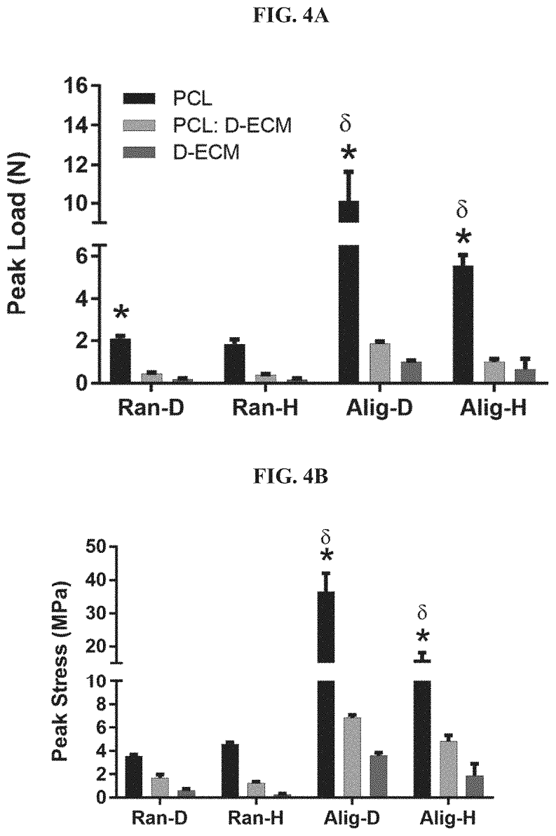

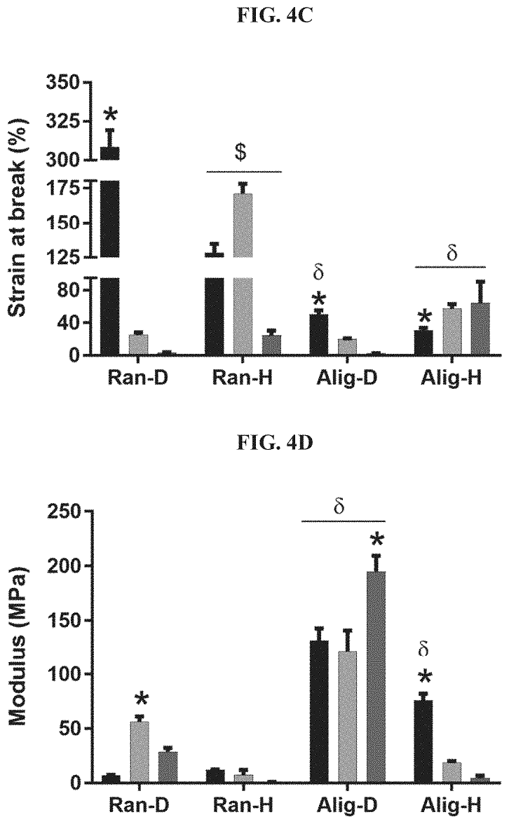

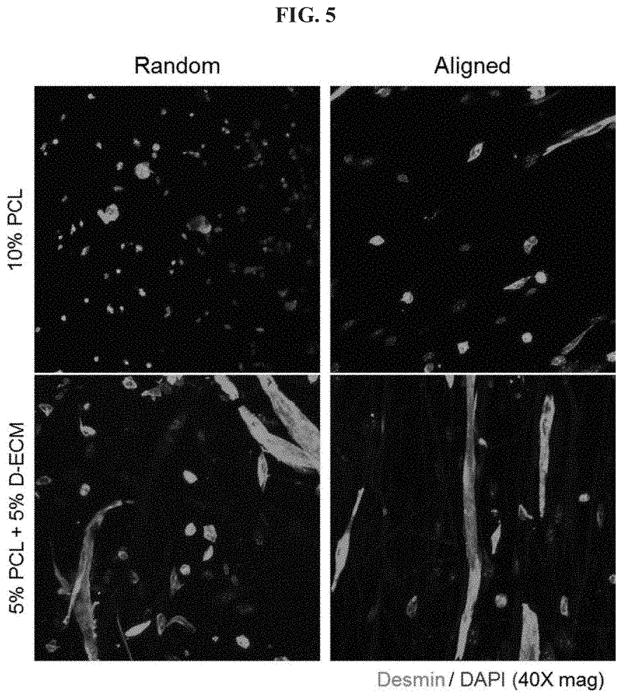

Uniaxial tensile testing was performed to failure on a scaffold. ‘Dog-bone’ shaped samples were cut from the electrospun scaffold (2.65 mm wide at their narrowest point with a gage length of 10 mm) and their thicknesses were measured with a Mitutoyo Absolute 547-500 micrometer (Mitutoyo America Corporation). Samples were then tested on an MTS Criterion Model 42 testing system with a 100N load cell (MTS Systems Corp.) at an extension rate of 10.0 mm/min Elastic modulus, peak stress and strain at break were calculated by the MTS software (MTS TestSuite: TW Elite) and recorded. The results are shown in FIG. 4. The pure PCL scaffolds exhibited the highest peak load and peak stress under both dry and hydrated conditions. However, statistical significance was not observed in all cases (FIG. 5). The anisotropic PCL scaffolds showed statistically higher peak stress, peak load and modulus but lower strain at break compared to the isotropic PCL scaffolds under both dry and hydrated conditions. Under dry conditions, all aligned scaffolds exhibited higher modulus compared to the random scaffolds. The aligned D-ECM scaffold had the highest modulus under dry conditions which correlated with the lowest strain at break, peak stress and peak load under dry conditions, indicating a brittle scaffold structure. The pure D-ECM scaffolds lost their structural integrity under hydrated conditions and had to be cross-linked before mechanical testing. The chemical cross-linking might explain the unexpected increase in strain at break and decrease in modulus of the D-ECM scaffolds under hydrated conditions. Overall, the addition of PCL improved the mechanical properties of the pure D-ECM scaffolds. The mechanical properties of the PCL:D-ECM blend scaffold were in between those of PCL and D-ECM scaffolds. Following mechanical characterization, the pure D-ECM group was removed from the study, due to its brittleness and poor structural integrity.

### Example 3

In this Example, cell response to electrospun scaffolds was determined.

Primary satellite cells were isolated from the hindlimb muscles of BALB/c mice and seeded at a density of 45,000 cells/well on 10 mm discs of electrospun scaffolds of 100% PCL, 100% D-ECM and PCL:D-ECM (50:50) in a 48 well plate. Cell seeded scaffolds were stained with desmin (abcam) and DAPI on day 4 of culture and imaged using confocal microscopy. The satellite cells were randomly distributed on the scaffolds with randomly oriented fibers. The cells were aligned end-to end on electrospun scaffolds with aligned fibers and some were found fused into multinucleated desmin⁺ myotubes. The hybrid 5% PCL and 5% D-ECM scaffolds had more myotubes that appeared thicker in diameter (FIG. 5). The pure 10% D-ECM scaffolds shrunk in culture media and disintegrated during the staining procedure.

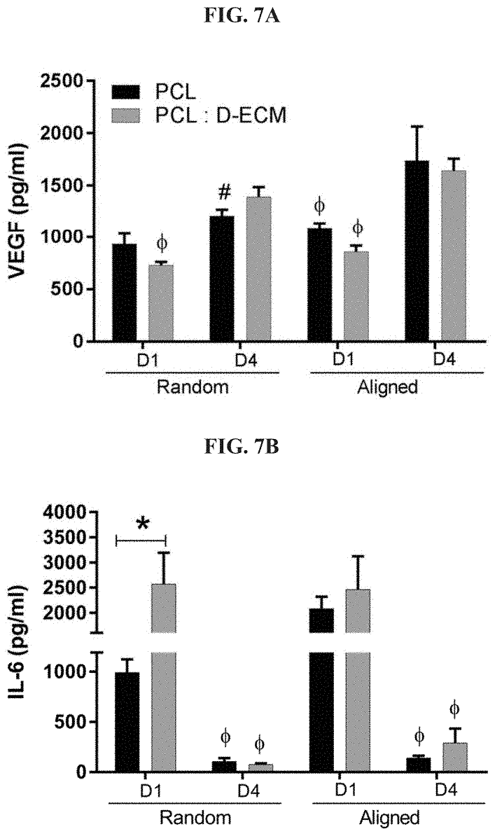

The VEGF and IL-6 production was quantified in cell culture supernatants collected on days 1 and 4. Overall, the VEGF production trended higher while the IL-6 production decreased from day 1 to 4 (FIG. 7). IL-6 production was significantly lower by day 4 in all groups (FIG. 7B). No significant differences were noted between random and aligned scaffolds except in VEGF production on PCL at day 4. The PCL and the PCL:D-ECM scaffolds also showed no statistical differences except in IL-6 production on day 1.

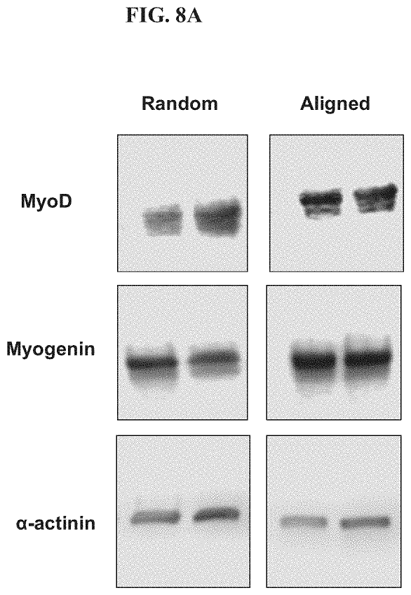

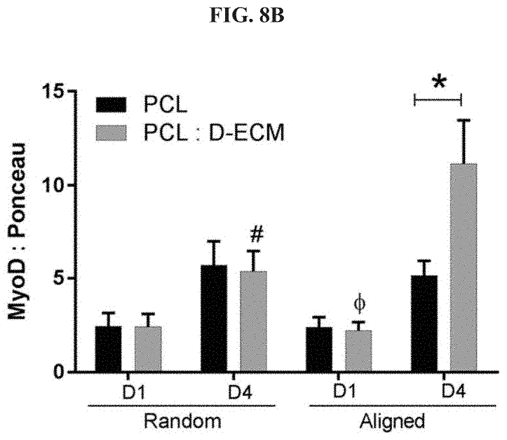

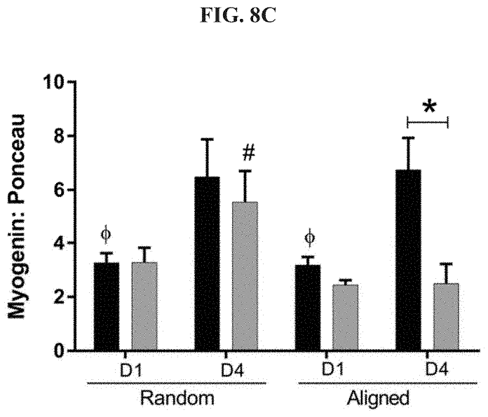

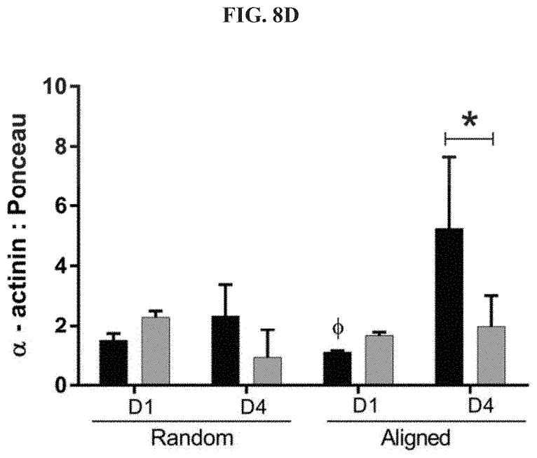

Cellular protein lysates were subjected to western blotting and probed for myogenic proteins (FIG. 8). MyoD expression, an indicator of early myogenic proliferation, was significantly higher by day 4 in the aligned PCL:D-ECM compared to the aligned PCL (FIG. 8B), which correlated with the histological images in FIG. 5D. Conversely, the expression of myogenin, a marker for late stage myoblast differentiation, was significantly lower on the aligned PCL:D-ECM compared to the aligned PCL scaffold (FIG. 8C). The only difference between random and aligned scaffolds were noted in the PCL:D-ECM blend in terms of MyoD and myogenin expression on day 4. The expression of MyoD increased on the aligned PCL:D-ECM blend from day 1 to 4 while the expression of myogenin increased on both the random and aligned PCL scaffold from day 1 to 4. The aligned PCL group also showed a significant increase in α-actinin expression from day 1 to day 4 as well as a significant increase compared to the aligned PCL:D-ECM scaffold at day 4 (FIG. 8D).

The D-ECM matrix of the present disclosure is solubilized and electrospun to create scaffolds with aligned fibers that mimic the native architecture of skeletal muscle. The D-ECM matrix of the present disclosure can also be electrospun to create scaffolds with randomly oriented fibers. The D-ECM matrix scaffold created using this technique has biologically relevant ratios of proteins and mimics both the native tissue composition and architecture. The D-ECM matrix scaffold can be used in a number of tissue engineering applications as an acellular scaffold (i.e., skeletal muscle regeneration, wound healing, stem cell expansion, etc). The D-ECM matrix scaffold can also be combined with cells to provide a substrate for cell growth, proliferation, and migration. The D-ECM matrix scaffold combined with cells can be used in a number of tissue engineering applications as a scaffold (i.e., skeletal muscle regeneration, wound healing, stem cell expansion, etc). The D-ECM matrix can also be combined with synthetic polymers to vary the mechanical properties of the D-ECM matrix scaffold.

What is claimed is:

1. An electrospun decellularized-extracellular muscle matrix (D-ECM matrix) fiber comprising: a D-ECM matrix.

2. The fiber of claim 1 comprising a diameter ranging from about 50 nanometers to about 2 μm.

3. The fiber of claim 1 further comprising a polymer.

4. The fiber of claim 3, wherein the polymer is chosen from polycaprolactone, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol) (PVA), poly(acrylic acid), poly(vinyl acetate), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), poly(vinyl acetate), polyvinylhydroxide, poly(ethylene oxide) (PEO) and polyorthoesters, and combinations thereof.

5. The fiber of claim 3, wherein the polymer is biodegradable.

6. A decellularized-extracellular muscle matrix (D-ECM matrix) scaffold comprising: an electrospun D-ECM matrix fiber.

7. The D-ECM matrix scaffold of claim 6, further comprises a polymer.

8. The D-ECM matrix scaffold of claim 6, wherein the D-ECM matrix fiber diameter ranges from about 50 nanometers to about 2 μm.

9. The D-ECM matrix scaffold of claim 6, wherein the fibers are aligned.

10. The D-ECM matrix scaffold of claim 6, wherein the fibers are randomly oriented.

11. The D-ECM matrix scaffold of claim 6, further comprising a cell.

12. A method for preparing a decellularized-extracellular muscle matrix (D-ECM matrix) scaffold, the method comprising: decellularizing a muscle tissue to prepare a D-ECM matrix; and electrospinning the D-ECM matrix to prepare a D-ECM matrix scaffold.

13. The method of claim 12, wherein the D-ECM matrix scaffold further comprises a polymer material.

14. The method of claim 12, wherein the polymer material is chosen from polycaprolactone, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol) (PVA), poly(acrylic acid), poly(vinyl acetate), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), poly(vinyl acetate), polyvinylhydroxide, poly(ethylene oxide) (PEO) and polyorthoesters, and combinations thereof.

15. The method of claim 13, wherein the polymer material is biodegradable.

16. The method of claim 12, wherein the electrospinning comprises: electrically charging a solution comprising a D-ECM matrix; and discharging the electrically charged solution onto a grounded target under an electrostatic field such that movement of the electrically charged solution under the electric field causes the electrically charged solution to evaporate and produce fibers of the D-ECM matrix on the grounded target.

17. The method of claim 12, further comprising seeding the D-ECM matrix scaffold with a cell.
